(19) United States
(12) Patent Application Publication
(10) Pub. No.: US 2023/0272319 A1
(43) Pub. Date: Aug. 31, 2023

(54) EXPOSURE UNIT, OBSERVATION DEVICE, AND EXPOSURE METHOD

(71) Applicant: HAMAMATSU PHOTONICS K.K., Hamamatsu-shi, Shizuoka (JP)

(72) Inventor: Yuji KIMURA, Hamamatsu-shi, Shizuoka (JP)

(73) Assignee: HAMAMATSU PHOTONICS K.K., Hamamatsu-shi, Shizuoka (JP)

(21) Appl. No.: 18/015,791

(22) PCT Filed: May 28, 2021
(86) PCT No.: PCT/JP2021/020509

§ 371 Date: Jan. 12, 2023

(30) Foreign Application Priority Data
Aug. 4, 2020 (JP) 2020-132338

(51) Int. Cl. C12M 1/00

(52) U.S. Cl. CPC C12M 23/02

(57) ABSTRACT

An exposure unit is used in order to expose a gas-phase substance to a cell in a buffer solution. The exposure unit includes a base body. The base body has a groove formed in the base body so as to be exposed to an outside and retaining the cell together with the buffer solution, and a first storage portion connected to one end of the groove and storing the buffer solution to be supplied to the groove.

### TECHNICAL FIELD

One aspect of the present disclosure relates to an exposure unit, an observation device, and an exposure method.

### BACKGROUND ART

Patent Literature 1 describes a gas-liquid interface culture method. In the gas-liquid interface culture method described in Patent Literature 1, cells are cultured on a support while a culture solution is supplied via the support from the back side of the support. The cells on the support are covered with a thin layer of the culture solution. This liquid layer is extremely thin, and thus a gas-phase substance can be brought into contact with the cells via the liquid layer. According to such a method, the effect of a gas-phase substance on cells can be evaluated.

### CITATION LIST

### Patent Literature

Patent Literature 1: Japanese Unexamined Patent Publication No. 2002-101897

### SUMMARY OF INVENTION

### Technical Problem

In a case where a gas-phase substance is exposed to cells in a buffer solution using the method described above, the liquid layer covering the cells needs to be kept thin in order to satisfactorily bring the substance into contact with the cells via the buffer solution. Meanwhile, for cell drying prevention, the cells need to be more reliably covered with the buffer solution. In other words, when the gas-phase substance is exposed to the cells in the buffer solution, it is required to keep the liquid layer of the buffer solution covering the cells at a suitable thickness.

An object of one aspect of the present disclosure is to provide an exposure unit, an observation device, and an exposure method allowing the liquid layer of a buffer solution covering cells to be kept at a suitable thickness when a gas-phase substance is exposed to the cells in the buffer solution.

### Solution to Problem

An exposure unit according to one aspect of the present disclosure is used in order to expose a gas-phase substance to a cell in a buffer solution. The exposure unit includes a base body. The base body has: a groove formed in the base body so as to be exposed to an outside and retaining the cell together with the buffer solution; and a first storage portion connected to one end of the groove and storing the buffer solution to be supplied to the groove.

In this exposure unit, when a suspension of the cell and the buffer solution is disposed in the groove, the cell is retained in the groove together with the buffer solution and the liquid layer of the buffer solution covering the cell is formed. The thickness of this liquid layer is a constant thickness in accordance with the configuration (for example, dimensions, hydrophilicity) of the groove. Accordingly, in the exposure unit, the liquid layer can be kept thin. In addition, the base body has the first storage portion connected to the one end of the groove and storing the buffer solution to be supplied to the groove. As a result, the buffer solution can be supplied from the first storage portion to the groove and drying of the cell can be prevented. As a result, according to the exposure unit, when the gas-phase substance is exposed to the cell in the buffer solution, the liquid layer of the buffer solution covering the cell can be kept at a suitable thickness.

The base body may include a first member having a surface where the groove is formed and a second member disposed on the surface, a first opening and a second opening may be formed in the second member, and the groove may be exposed to the outside via the first opening and at least a part of the first storage portion may be configured by the second opening. In this case, the groove is exposed to the outside via the first opening, and thus the suspension of the cell and the buffer solution can be easily disposed in the groove. In addition, since at least a part of the first storage portion is configured by the second opening, a large storage amount of the buffer solution in the first storage portion can be ensured.

A plurality of the first openings may be formed in the second member. In this case, the exposing area of the groove can be increased and the exposure flow rate can be increased. In addition, the cell disposed at the part of the groove corresponding to one of the first openings can be different from the cell disposed at the part of the groove corresponding to another first opening, and a plurality of types of cells can be exposed at once.

A part of the base body where the groove is formed may be configured to be transparent with respect to light from the cell retained in the groove. In this case, the light from the cell can be observed via the base body.

The base body may further include a second storage portion connected to the other end of the groove and storing the buffer solution discharged from the groove. In this case, the buffer solution discharged from the groove can be stored in the second storage portion.

Hydrophilicity of an inner surface of the groove may be higher than hydrophilicity of a part of the base body adjacent to the groove. In this case, the cell can be easily retained in the groove.

The groove may include a meanderingly extending part. In this case, the area of the groove can be increased and the exposure flow rate can be increased.

The first storage portion may be configured by a recess formed in the base body. In this case, the configuration can be simplified.

An observation device according to one aspect of the present disclosure includes: the exposure unit; an optical system through which light from the cell retained in the groove passes; and a photodetector detecting the light that has passed through the optical system. According to this observation device, for the reasons described above, when the gas-phase substance is exposed to the cell in the buffer solution, the liquid layer of the buffer solution covering the cell can be kept at a suitable thickness. As a result, observation can be performed satisfactorily.

An exposure unit according to one aspect of the present disclosure is used in order to expose a gas-phase substance to a cell in a buffer solution. The exposure unit includes a base body. A surface of the base body includes a hydrophilic region and a hydrophobic region less hydrophilic than the hydrophilic region. By partitioning the surface of the base body into the hydrophilic region and the hydrophobic region, the hydrophilic region includes a cell holding portion where the cell is retained together with the buffer solution and a storage portion connected to the cell holding portion and storing the buffer solution to be supplied to the cell holding portion.

In this exposure unit, when a suspension of the cell and the buffer solution is disposed in the cell holding portion, the cell is retained in the cell holding portion together with the buffer solution and the liquid layer of the buffer solution covering the cell is formed. The thickness of this liquid layer is a constant thickness in accordance with the configuration (for example, dimensions, hydrophilicity) of the cell holding portion. Accordingly, in the exposure unit, the liquid layer can be kept thin. In addition, the hydrophilic region has the storage portion connected to the cell holding portion and storing the buffer solution to be supplied to the cell holding portion. As a result, the buffer solution can be supplied from the storage portion to the cell holding portion and drying of the cell can be prevented. As a result, according to the exposure unit, when the gas-phase substance is exposed to the cell in the buffer solution, the liquid layer of the buffer solution covering the cell can be kept at a suitable thickness.

An exposure method according to one aspect of the present disclosure is for exposing the gas-phase substance to the cell retained in the groove using the exposure unit described above in a state where the cell is retained in the groove together with the buffer solution and the buffer solution to be supplied to the groove is stored in the first storage portion. According to this exposure method, for the reasons described above, when the gas-phase substance is exposed to the cell in the buffer solution, the liquid layer of the buffer solution covering the cell can be kept at a suitable thickness.

An exposure method according to one aspect of the present disclosure is for exposing the gas-phase substance to the cell retained in the cell holding portion using the exposure unit described above in a state where the cell is retained in the cell holding portion together with the buffer solution and the buffer solution to be supplied to the cell holding portion is stored in the storage portion. According to this exposure method, for the reasons described above, when the gas-phase substance is exposed to the cell in the buffer solution, the liquid layer of the buffer solution covering the cell can be kept at a suitable thickness.

### Advantageous Effects of Invention

According to one aspect of the present disclosure, it is possible to provide an exposure unit, an observation device, and an exposure method allowing the liquid layer of a buffer solution covering cells to be kept at a suitable thickness when a gas-phase substance is exposed to the cells in the buffer solution.

### BRIEF DESCRIPTION OF DRAWINGS

FIG. 1 is a perspective view of an exposure unit of an embodiment.

FIG. 2 is an exploded perspective view of the exposure unit.

FIG. 3(a) is a plan view of a glass substrate, and FIG. 3(b) is a cross-sectional view taken along line B-B illustrated in FIG. 3(a).

FIG. 4(a) is a perspective view illustrating how a buffer solution is perfused, and FIG. 4(b) is a cross-sectional view taken along line B-B illustrated in FIG. 4(a).

FIGS. 5(a) to 5(c) are cross-sectional views illustrating how cells are seeded.

FIG. 6 is a cross-sectional view illustrating how the buffer solution is supplied.

FIGS. 7(a) and 7(b) are diagrams illustrating a state where a gas sample chamber is disposed on the exposure unit.

FIG. 8 is a diagram illustrating a state during fluorescence observation.

FIG. 9 is a cross-sectional view illustrating how odor molecules come into contact with the cells.

FIG. 10 is an exploded perspective view of an exposure unit of a first modification example.

FIG. 11(a) is a plan view of a glass substrate of the first modification example, and FIG. 11(b) is a plan view of a rubber sheet of the first modification example.

FIG. 12(a) is a plan view of a glass substrate of a second modification example, FIG. 12(b) is a cross-sectional view taken along line B-B illustrated in FIG. 12(a), and FIG. 12(c) is a cross-sectional view taken along line C-C illustrated in FIG. 12(a).

FIGS. 13(a) to 13(c) are cross-sectional views illustrating how cells are seeded in the second modification example.

FIGS. 14(a) and 14(b) are cross-sectional views illustrating how a buffer solution is supplied in the second modification example.

FIGS. 15(a) and 15(b) are diagrams illustrating a state where an exposure unit of the second modification example is disposed in a gas sample chamber.

FIG. 16(a) is a plan view of an exposure unit of a second embodiment, and FIG. 16(b) is a cross-sectional view taken along line B-B illustrated in FIG. 16(a).

FIGS. 17(a) to (c) are cross-sectional views illustrating how cells are seeded in the second embodiment.

FIGS. 18(a) and 18(b) are cross-sectional views illustrating how a buffer solution is supplied in the second embodiment.

FIG. 19 is a diagram illustrating a state during fluorescence observation using the exposure unit of the second embodiment.

FIG. 20 is a cross-sectional view taken along line XX-XX illustrated in FIG. 19.

### DESCRIPTION OF EMBODIMENTS

Hereinafter, an embodiment of the present disclosure will be described in detail with reference to the drawings. In the following description, the same reference numerals are used for the same or corresponding elements with redundant description omitted.

### First Embodiment

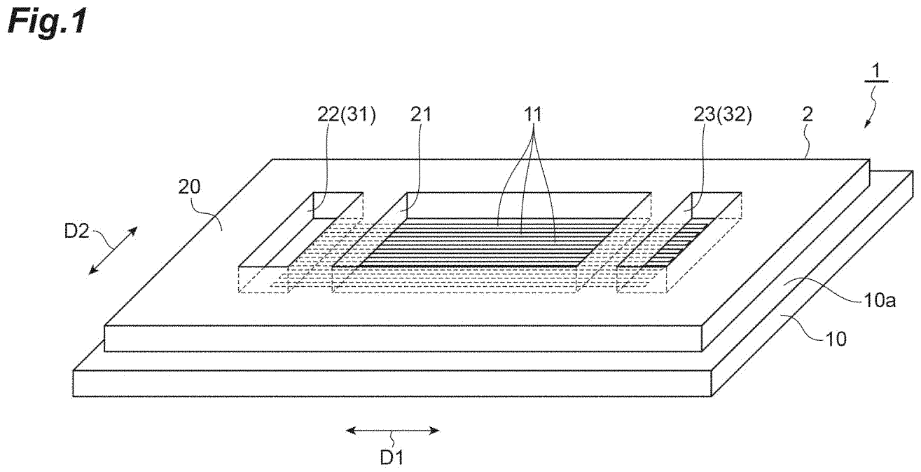

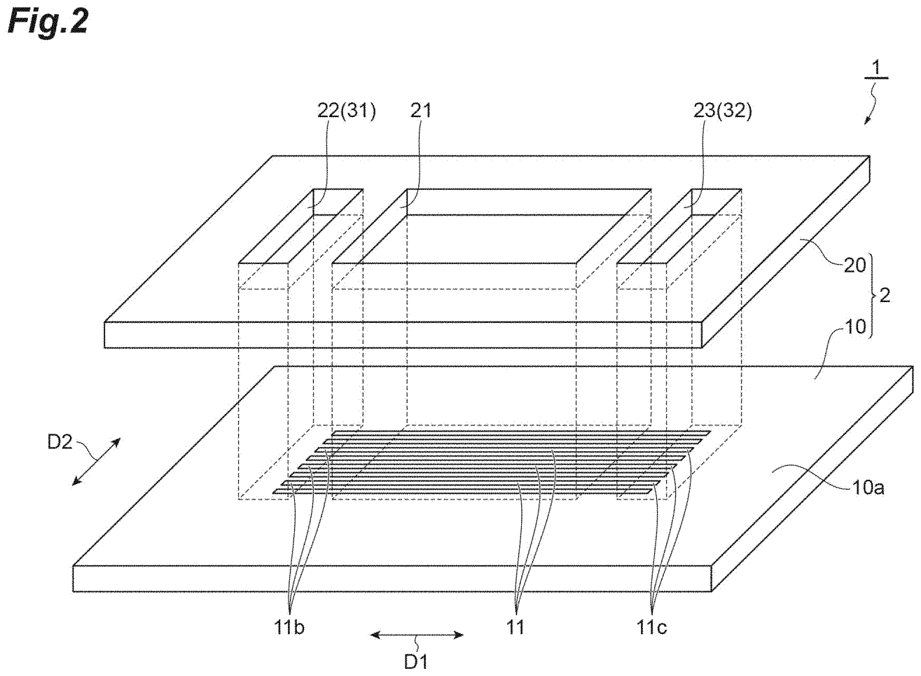

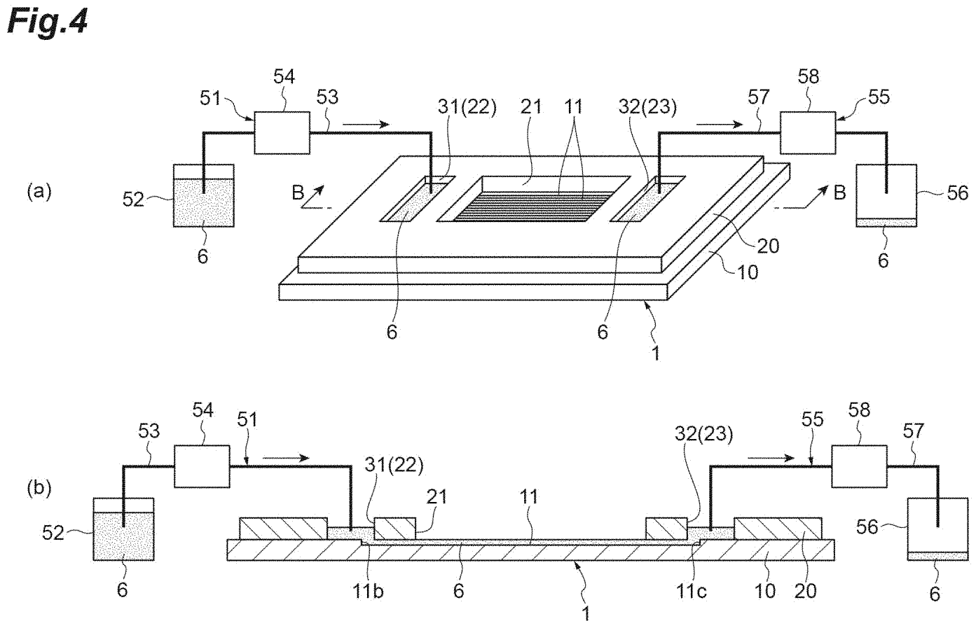

An exposure unit 1 illustrated in FIGS. 1 to 3 is used in order to expose gas-phase substances 5 to cells 7 in a buffer solution 6 (FIGS. 4 to 9). How to use the exposure unit 1 will be described later. The exposure unit 1 configures a part of an observation device 50, which will be described later. First, the configuration of the exposure unit 1 will be described below.

### [Exposure Unit]

As illustrated in FIGS. 1 to 3, the exposure unit 1 includes a base body 2. The base body 2 has a glass substrate (first member) 10 and a rubber sheet (second member) 20. The glass substrate 10 is, for example, formed of glass in a rectangular plate shape and has light transmittance. The glass substrate 10 has a flat surface 10a.

The surface 10a of the glass substrate 10 has a plurality of grooves 11. Each groove 11 linearly extends along a first direction D1. The plurality of grooves 11 are arranged at regular intervals in a second direction D2 orthogonal to the first direction D1. The plurality of grooves 11 have the same shape. The length of the groove 11 in the extension direction (first direction D1) is, for example, 20 mm.

The groove 11 has a rectangular shape (square shape in this example) in a cross section perpendicular to the extension direction. The width and depth of the groove 11 are, for example, 20 μm each. The width and depth of the groove 11 are set such that the cells 7 can be sufficiently accommodated in the grooves 11 and, when the cells 7 are accommodated in the grooves 11, the cells 7 are sufficiently covered with the liquid layer of the buffer solution 6. The width of the groove 11 is set to, for example, 10 μm to 200 μm. The depth of the groove 11 is set to, for example, 10 μm to 100 μm. The groove 11 is formed using, for example, a micro-machining technique such as etching.

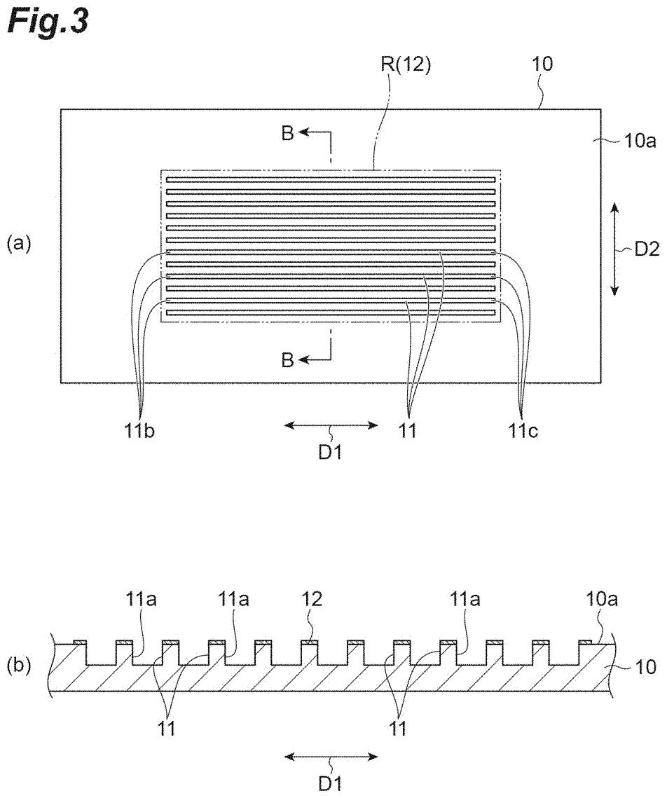

The part of the surface 10a of the glass substrate 10 adjacent to the grooves 11 is hydrophobically treated. In this example, the inside of a region R illustrated in FIG. 3(a) is hydrophobically treated. The region R is set so as to surround the plurality of grooves 11. In this example, a layered hydrophobic pattern 12 made of a hydrophobic coating is formed on the surface 10a in the region R. The hydrophobic pattern 12 is not formed on inner surfaces 11a of the grooves 11. As a result, the hydrophilicity of the inner surfaces 11a of the grooves 11 is higher than the hydrophilicity of the part of the glass substrate 10 adjacent to the grooves 11 (hydrophobically treated part). In other words, the hydrophobicity (water repellency) of the part of the glass substrate 10 adjacent to the groove 11 is higher than the hydrophobicity (water repellency) of the inner surface 11a of the groove 11.

The rubber sheet 20 is, for example, formed of silicone rubber in a rectangular plate shape. The rubber sheet 20 is configured by, for example, polydimethylsiloxane (PDMS). The rubber sheet 20 is disposed on the surface 10a of the glass substrate 10 and is in close contact with the surface 10a.

A first opening 21, a second opening 22, and a third opening 23 are formed in the rubber sheet 20. Each of the openings 21, 22, and 23 penetrates the rubber sheet 20 along the thickness direction. The first opening 21 is formed in a rectangular shape and disposed in the middle portion of the rubber sheet 20. The second opening 22 and the third opening 23 are formed in the same rectangular shape and are disposed on both sides of the first opening 21 in the first direction D1. Each of the lengths of the second opening 22 and the third opening 23 along the first direction D1 is shorter than the length of the first opening 21 along the first direction D1. Each of the lengths of the second opening 22 and the third opening 23 along the second direction D2 is equal to the length of the first opening 21 along the second direction D2.

The rubber sheet 20 is disposed on the glass substrate 10 such that the first opening 21 overlaps the middle portion of each groove 11. As a result, the middle portion of each groove 11 is exposed to the outside via the first opening 21. In addition, in a state where the rubber sheet is disposed on the glass substrate 10, the second opening 22 overlaps one end 11b of each groove 11 and the third opening 23 overlaps the other end 11c of each groove 11.

The second opening 22 configures a first storage portion 31 for storing the buffer solution 6 to be supplied to the groove 11. The first storage portion 31 is configured by the space in the second opening 22 defined by the inner surface of the second opening 22 and a part of the surface 10a of the glass substrate 10. The first storage portion 31 is connected to the one end 11b of each groove 11. The third opening 23 configures a second storage portion 32 for storing the buffer solution 6 discharged from the groove 11. The second storage portion 32 is configured by the space in the third opening 23 defined by the inner surface of the third opening 23 and a part of the surface 10a of the glass substrate 10. The second storage portion 32 is connected to the other end 11c of each groove 11.

### [Observation Device]

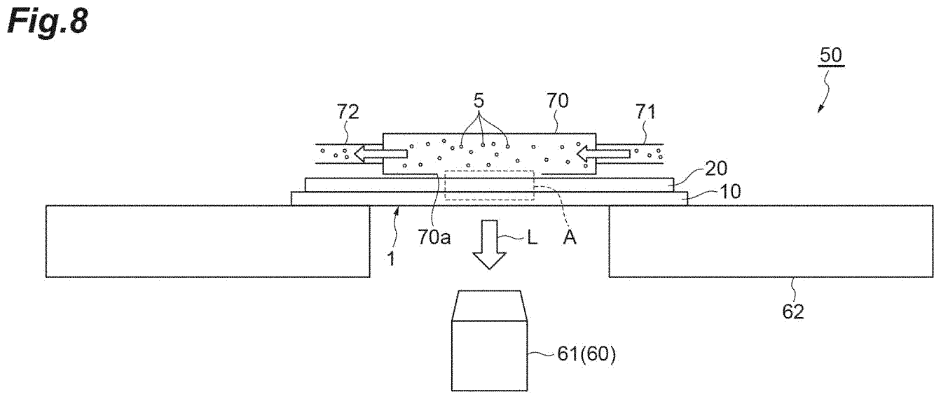

As described above, the exposure unit 1 configures a part of the observation device 50 (FIG. 8). In this example, the observation device 50 is a fluorescence observation device for observing fluorescence emitted from the cells 7 while exposing the gas-phase substances 5 to the cells 7 in the buffer solution 6. The buffer solution 6 is an aqueous solution in which the cells 7 are dissolved. In order to operate the cell 7, the cell 7 needs to be dissolved in an aqueous solution.

The cell 7 is, for example, an odor detection cell and the fluorescence intensity thereof increases by exposure to an odor substance. In this case, the substance 5 is an odor substance. The odor detection cell is a cultured insect cell that expresses an insect olfactory receptor on the cell membrane (cell surface) and expresses green fluorescent protein GCaMP6s in the cytoplasm (intracellularly). When a certain low-molecular-weight compound such as geosmin, which is a musty-smelling substance, binds to the insect olfactory receptor, calcium ions flow into the cell from the outside of the cell through the insect olfactory receptor. When the calcium ions bind to GCaMP6s after the inflow, the fluorescence intensity of GCaMP6s increases. Accordingly, geosmin (odor substance) can be detected by monitoring the intensity of fluorescence emitted from the odor detection cell. In this manner, the observation device 50 can be used as a trace chemical detection device.

As illustrated in FIG. 4, the observation device 50 includes, in addition to the exposure unit 1, a supply unit 51 supplying the buffer solution 6 to the first storage portion 31 and a discharge unit 55 discharging the buffer solution 6 from the second storage portion 32. The supply unit 51 has a container 52, a flow path member 53, and a pump 54. The container 52 stores the buffer solution 6. The flow path member 53 is a member through which the buffer solution 6 flows and is configured by, for example, a tube. The pump 54 pressure-feeds the buffer solution 6 from the container 52 toward the first storage portion 31 via the flow path member 53.

The discharge unit 55 has a container 56, a flow path member 57, and a pump 58. The container 56 stores the buffer solution 6 discharged from the second storage portion 32. The flow path member 57 is a member through which the buffer solution 6 flows and is configured by, for example, a tube. The pump 58 pressure-feeds the buffer solution 6 from the second storage portion 32 toward the container 56 via the flow path member 57. The buffer solution 6 is supplied to and discharged from the groove 11 by the supply unit 51 and the discharge unit 55.

As illustrated in FIG. 8, the observation device 50 further includes an optical system 60 through which light L from the cell 7 passes. In this example, the light L is fluorescence emitted from the cell 7. The optical system 60 is configured to include an objective lens 61. The objective lens 61 is disposed so as to face the exposure unit 1 disposed on a microscope stage 62. In addition, the observation device 50 further includes a photodetector (not illustrated) detecting the light L that has passed through the optical system 60.

### [Observation Method]

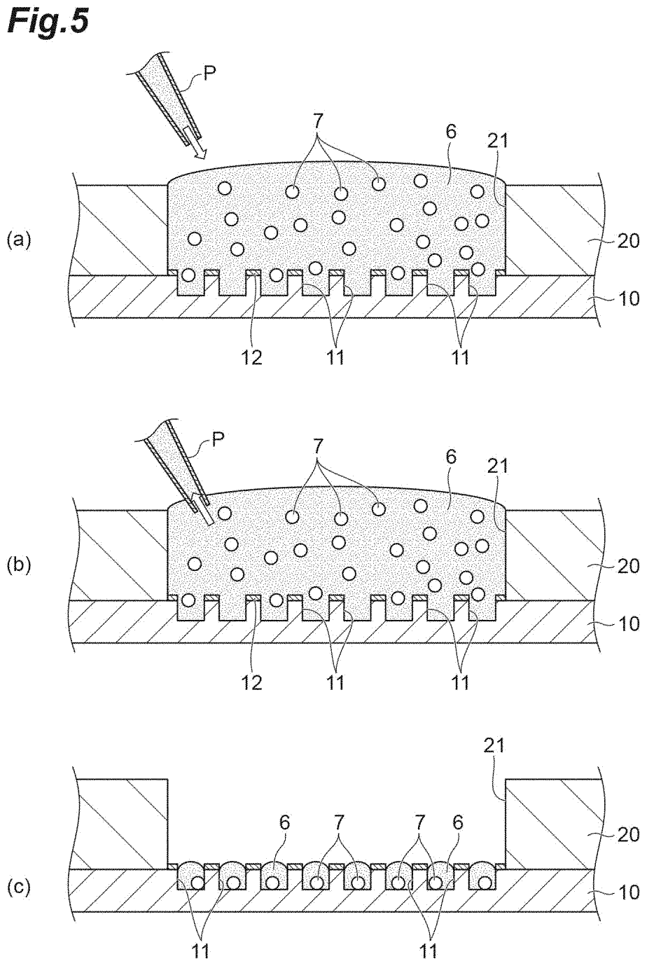

An example of a method for exposing the gas-phase substance 5 to the cell 7 in the buffer solution 6 and observing the light L from the cell 7 using the observation device 50 will be described with reference to FIGS. 5 to 8. In this example, the cell 7 is an Sf21 cell expressing an olfactory receptor and GCaMP6s. The buffer solution 6 is Ringer's solution (140 mM NaCl, 5.6 mM KCl, 4.5 mM CaCL₂, 11.26 mM MgCl₂, 10 mM HEPES, and 9.4 mM D-glucose, pH 7.2).

First, as illustrated in FIG. 5(a), a suspension of the buffer solution 6 and the cells 7 is dripped into the first opening 21 with a pipette P. Subsequently, as illustrated in FIG. 5(b), the dripped suspension is suctioned out with the pipette P and removed. At this time, surface tension causes some of the suspension to remain in the grooves 11 as illustrated in FIG. 5(c). The cells 7 settle in the respective grooves 11 and are seeded at the positions corresponding to first opening 21 in the respective grooves 11. The settled cells 7 are covered with the buffer solution 6. In other words, the cells 7 are retained in the grooves 11 together with the buffer solution 6, and a liquid layer of the buffer solution 6 covering the cells 7 is formed. It should be noted that instead of suctioning out the suspension, only the buffer solution 6 may be suctioned out after waiting for the cells 7 to settle.

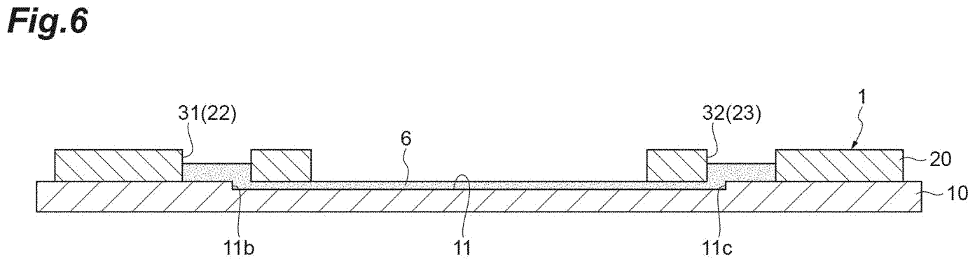

Subsequently, the buffer solution 6 is dripped into the first storage portion 31 as illustrated in FIG. 6. Since the first storage portion 31 is connected to the grooves 11, the buffer solution 6 moves along the grooves 11 from the first storage portion 31 and is supplied to the cells 7 staying at the positions corresponding to the first opening 21 in the respective grooves 11. Some of the buffer solution 6 may move along the grooves 11 and reach the second storage portion 32.

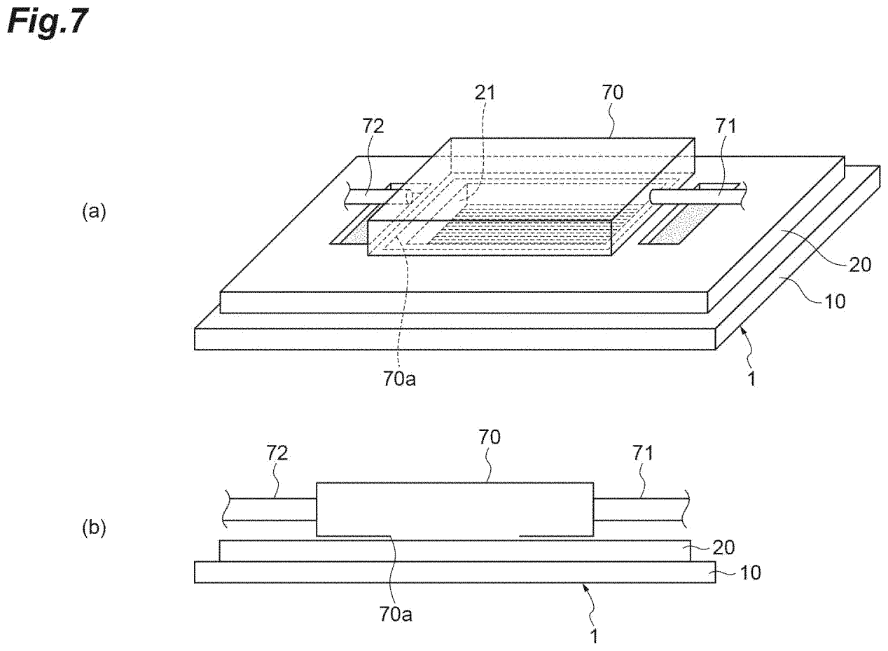

Subsequently, a gas sample chamber 70 is disposed on the exposure unit 1 as illustrated in FIGS. 7(a) and 7(b). An opening 70a is formed in the bottom surface of the gas sample chamber 70. The gas sample chamber 70 is brought into close contact with the exposure unit 1 such that the opening 70a is connected to the first opening 21. An inflow pipe 71 for gas inflow is connected to one side surface of the gas sample chamber 70, and a discharge pipe 72 for gas discharge is connected to the other side surface of the gas sample chamber 70. The inflow pipe 71 and the discharge pipe 72 are configured by, for example, tubes. The upper surface of the gas sample chamber 70 (surface on the side opposite to the bottom surface) is configured to be transparent and capable of transmitting illumination light.

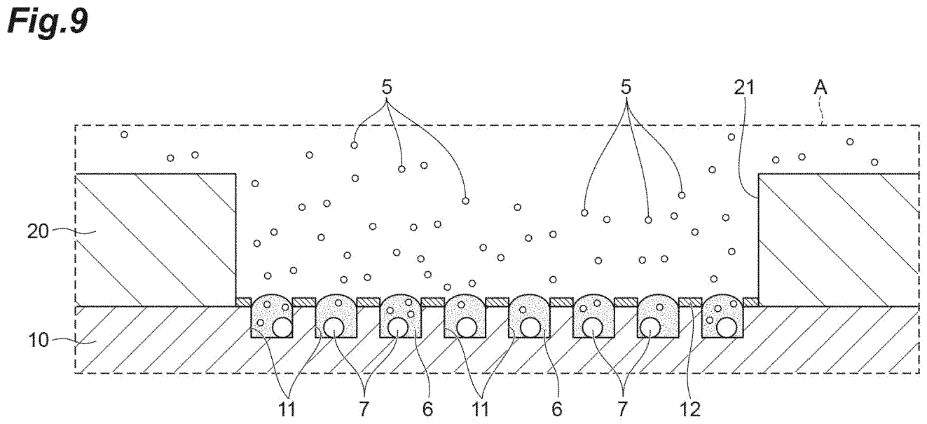

Subsequently, as illustrated in FIGS. 8 and 9, the exposure unit 1 is installed on the microscope stage 62 and observation is started. In FIG. 9, the part A illustrated in FIG. 8 is illustrated in an enlarged manner. When the observation is started, the pump 54 of the supply unit 51 and the pump 58 of the discharge unit 55 are driven to start the supply of the buffer solution 6 from the first storage portion 31 to the groove 11 and the discharge of the buffer solution 6 from the groove 11 to the second storage portion 32. The detection of the light L emitted from the cell 7 is started, and the intensity of the light L at the observation initiation is measured as a baseline. The light L from the cell 7 is detected by the above photodetector via the objective lens 61. The light L from the cell 7 retained in the groove 11 is transmitted through the glass substrate 10 and enters the objective lens 61. In other words, in the observation device 50, the glass substrate 10 is formed of glass and configured to be transparent with respect to the light L, and the light L is observed via the glass substrate 10.

Subsequently, a gas containing the substance 5, which is an odor substance, is caused to flow into the gas sample chamber 70 via the inflow pipe 71. The gas containing the substance 5 is discharged from the gas sample chamber 70 via the discharge pipe 72. When the gas containing the substance 5 reaches the first opening 21, the substance 5 comes into contact with the cell 7 via the liquid layer of the buffer solution 6 in each groove 11 and the substance 5 is exposed to the cell 7. As a result, the fluorescence intensity of the cell 7 increases. By comparing the detected fluorescence intensity with the intensity measured as the baseline, the degree of rise in the fluorescence intensity of the cell 7 can be grasped.

In this manner, in the observation method (exposure method) using the observation device 50, the gas-phase substance 5 is exposed to the cell 7 retained in the groove 11 in a state where the cell 7 is retained in the groove 11 together with the buffer solution 6 and the buffer solution 6 to be supplied to the groove 11 is stored in the first storage portion 31. Although the amount of the buffer solution 6 held in the grooves 11 is small and thus the cells 7 are likely to dry out, by continuously supplying the buffer solution 6 from the first storage portion 31, the cells 7 are prevented from drying out and the response of the cells 7 to the substances can be continuously observed while maintaining the activity of the cells 7.

It should be noted that the buffer solution 6 may be perfused by the pressure attributable to the head difference between the first storage portion 31 and the second storage portion 32 instead of perfusing (supplying and discharging) the buffer solution 6 with respect to the groove 11 with the supply unit 51 and the discharge unit 55. Alternatively, the buffer solution 6 may be perfused by disposing the exposure unit 1 so as to be tilted with respect to the horizontal direction such that the buffer solution 6 flows from the first storage portion 31 toward the second storage portion 32. The buffer solution 6 may be perfused by disposing an absorber such as a sponge in the outlet side end portion of the groove 11 (the other end 11c). In these cases, the supply unit 51 and the discharge unit 55 may not be provided. In a case where observation is performed for a limited time, the buffer solution 6 corresponding to the dried portion is supplied from the first storage portion 31, and thus the liquid layer of the buffer solution 6 covering the cells 7 is kept even without the supply unit 51 supplying the buffer solution 6 to the first storage portion 31. The same applies to modification examples and a second embodiment, which will be described later.

### [Action and Effect]

As described above, in the exposure unit 1, when the suspension of the cells 7 and the buffer solution 6 is disposed in the grooves 11, the cells 7 are retained in the grooves 11 together with the buffer solution 6 and the liquid layer of the buffer solution 6 covering the cells 7 is formed. The thickness of this liquid layer is a constant thickness in accordance with the configuration (for example, dimensions, hydrophilicity) of the grooves 11. Accordingly, in the exposure unit 1, the liquid layer can be kept thin. In other words, in the exposure unit 1, the dimensions (width, depth) and hydrophilicity (hydrophilicity of the surface 10a, hydrophilicity of the inner surface 11a of the groove 11) of the groove 11 are set such that the liquid layer of the buffer solution 6 covering the cell 7 has a desired thickness when the suspension of the cell 7 and the buffer solution 6 is disposed in the groove 11. The thickness of the liquid layer of the buffer solution 6 covering the cell 7 is adjusted to, for example, approximately 5 μm to 300 μm. In addition, the base body 2 has the first storage portion 31 connected to the one end 11b of the groove 11 and storing the buffer solution 6 to be supplied to the groove 11. As a result, the buffer solution 6 can be supplied from the first storage portion 31 to the groove 11 and drying of the cells 7 can be prevented. As a result, according to the exposure unit 1, when the gas-phase substance 5 is exposed to the cell 7 in the buffer solution 6, the liquid layer of the buffer solution 6 covering the cell 7 can be kept at a suitable thickness. In addition, according to the exposure unit 1, exposure of the substance 5 to the cell 7 can be facilitated. That is, although bringing an aqueous solution in which the substance 5 is dissolved into contact with the cell 7 is conceivable as a method for exposing the substance 5 to the cell 7, it takes time and effort to dissolve the substance 5 in the aqueous solution. In this regard, by the exposure method using the exposure unit 1, such time and effort can be omitted and exposure of the substance 5 to the cell 7 can be facilitated. In some existing methods without appropriate cell drying prevention, exposure is possible only for a short time so that cell drying is avoided. According to the exposure unit 1, drying of the cells 7 is appropriately prevented as described above, and thus such a situation can be suppressed.

In the exposure unit 1, the groove 11 is exposed to the outside via the first opening 21, and thus the suspension of the cell 7 and the buffer solution 6 can be easily disposed in the groove 11. In addition, since the first storage portion 31 is configured by the second opening 22, a large storage amount of the buffer solution 6 in the first storage portion 31 can be ensured.

The glass substrate 10 is configured to be transparent with respect to the light L from the cell 7 retained in the groove 11. As a result, the light L can be observed via the glass substrate 10.

The base body 2 has the second storage portion 32 connected to the other end 11c of the groove 11 and storing the buffer solution 6 discharged from the groove 11. As a result, the buffer solution 6 discharged from the groove 11 can be stored in the second storage portion 32.

The hydrophilicity of the inner surface 11a of the groove 11 is higher than the hydrophilicity of the part of the glass substrate 10 adjacent to the groove 11. As a result, the cells 7 can be easily retained in the grooves 11. In other words, the buffer solution 6 can be dammed by the hydrophobic pattern 12.

### MODIFICATION EXAMPLES

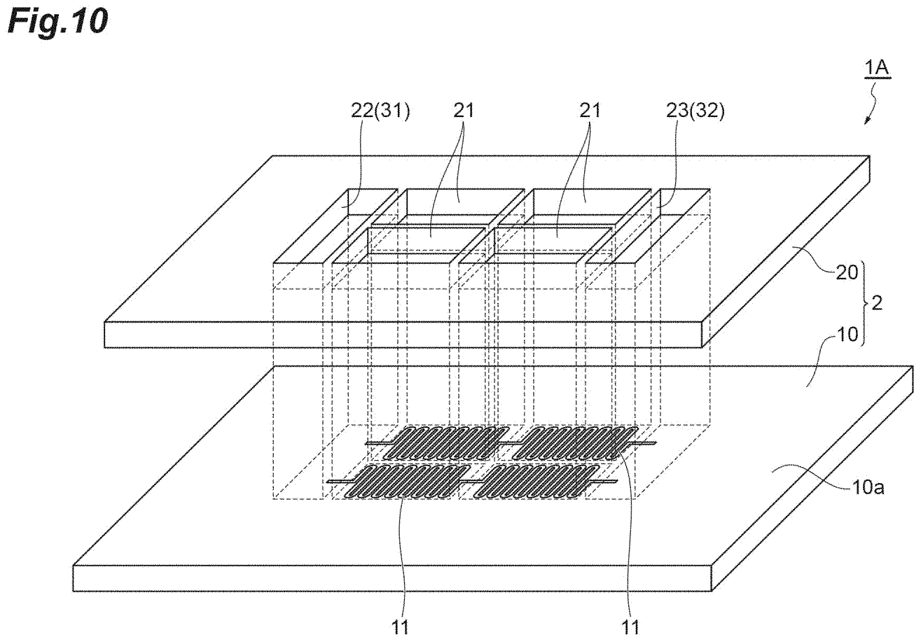

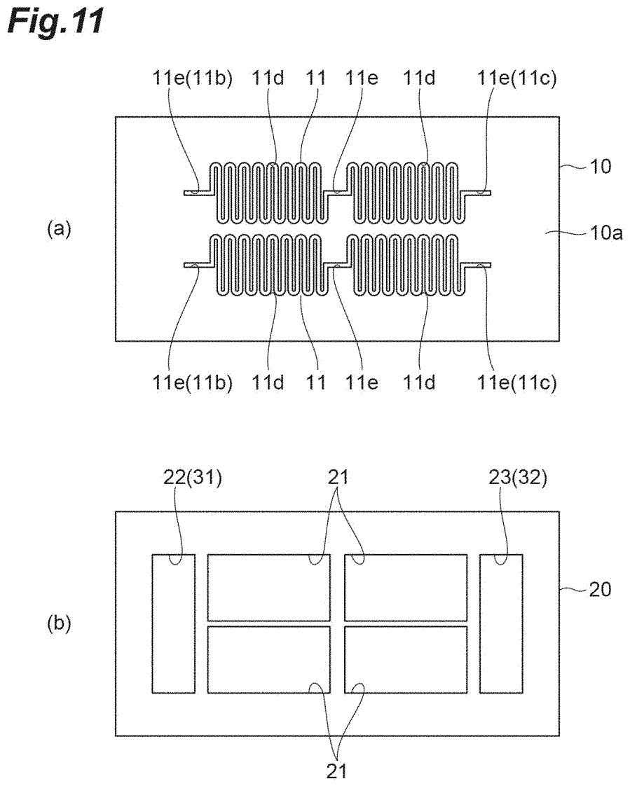

In an exposure unit 1A of a first modification example illustrated in FIGS. 10 and 11, the surface 10a of the glass substrate 10 has a pair of the grooves 11. Each groove 11 includes a plurality of meandering parts 11d (two in this example) and a plurality of linear parts 11e (three in this example). Each meandering part 11d extends meanderingly. In each groove 11, the two meandering parts 11d are connected by one of the linear parts 11e. The other two linear parts 11e configure the one end 11b and the other end 11c of the groove 11, respectively.

A plurality of the first openings 21 (four in this example) are formed in the rubber sheet 20. The plurality of first openings 21 are formed in the same rectangular shape and respectively overlap the meandering parts 11d. The first openings 21 are mutually separated by the rubber sheet 20.

According to the first modification example as well as the first embodiment, the liquid layer of the buffer solution 6 covering the cells 7 can be kept at a suitable thickness when the gas-phase substances 5 are exposed to the cells 7 in the buffer solution 6. In addition, the plurality of first openings 21 are formed in the rubber sheet 20. As a result, the exposing area of the groove 11 can be increased and the exposure flow rate can be increased. In addition, the cell 7 disposed at one meandering part 11d can be different from the cell 7 disposed at the other meandering part 11d, and a plurality of types of cells 7 can be exposed at once. In addition, since the groove 11 includes the meanderingly extending meandering part 11d, the area of the groove 11 can be increased and the exposure flow rate can be increased.

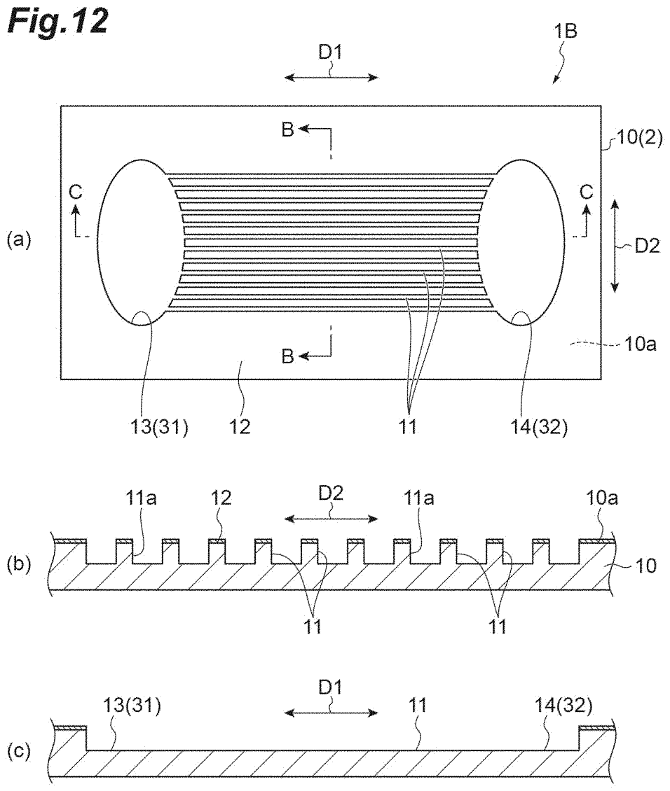

In an exposure unit 1B of a second modification example illustrated in FIG. 12, the base body 2 has only the glass substrate 10 and does not have the rubber sheet 20. The surface 10a of the glass substrate has a first recess 13 and a second recess 14 in addition to the plurality of grooves 11.

In a plan view (when viewed in the direction perpendicular to the surface 10a), each of the first recess 13 and the second recess 14 has, for example, an elliptical shape with a long axis along the second direction D2. Each of the first recess 13 and the second recess 14 has, for example, the same depth as the groove 11. Each of the first recess 13 and the second recess 14 is connected to each groove 11.

In the second modification example, the first recess 13 configures the first storage portion 31 and the second recess 14 configures the second storage portion 32. The hydrophobic pattern 12 is formed on the entire surface 10a of the glass substrate 10. As a result, the hydrophilicity of the inner surface 11a of the groove 11 is higher than the hydrophilicity of the part of the glass substrate 10 adjacent to the groove 11.

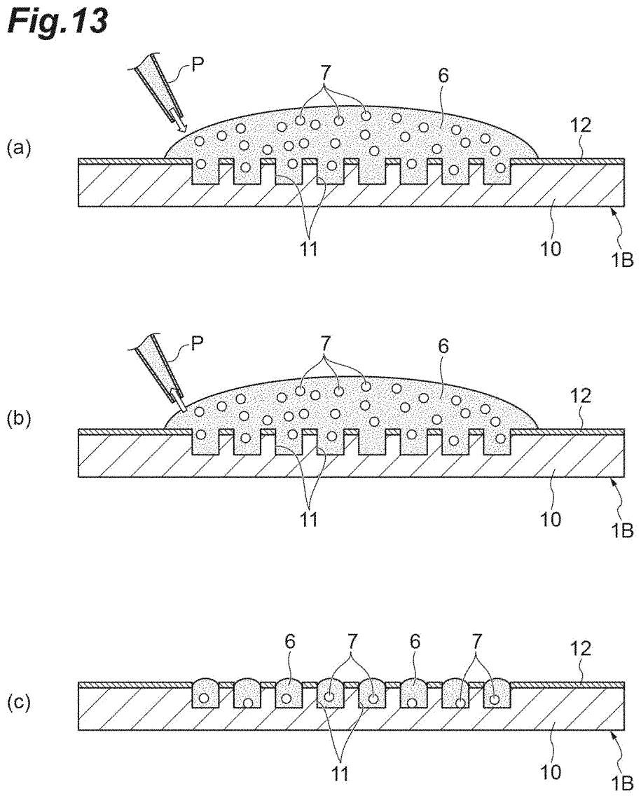

In an observation method using the exposure unit 1B, as illustrated in FIG. 13(a), a suspension of the buffer solution 6 and the cells 7 is dripped around the grooves 11 with the pipette P. Subsequently, as illustrated in FIG. 13(b), the dripped suspension is suctioned out with the pipette P and removed. At this time, surface tension causes some of the suspension to remain in the grooves 11 as illustrated in FIG. 13(c). The cells 7 settle in the respective grooves 11 and are seeded in the respective grooves 11. The settled cells 7 are covered with the buffer solution 6. In other words, the cells 7 are retained in the grooves 11 together with the buffer solution 6, and a liquid layer of the buffer solution 6 covering the cells 7 is formed.

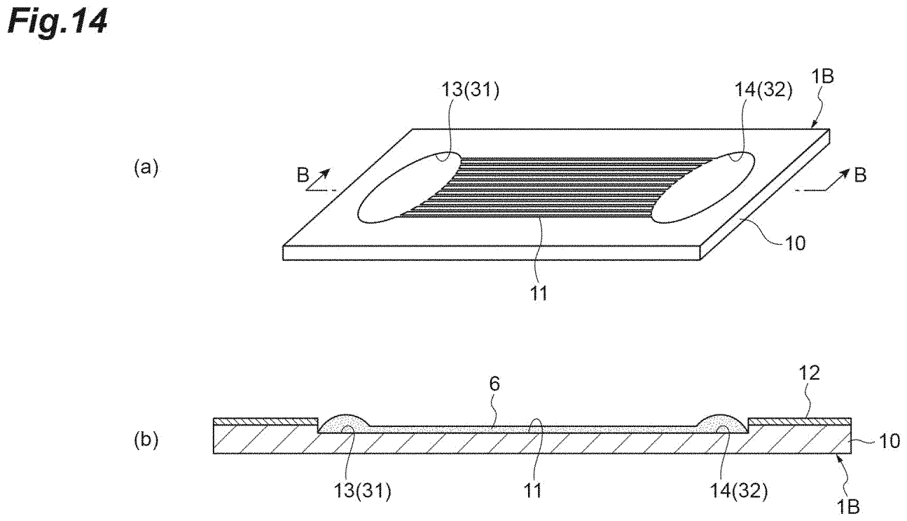

Subsequently, the buffer solution 6 is dripped into the first storage portion 31 (first recess 13) as illustrated in FIGS. 14(a) and 14(b). Since the first storage portion 31 is connected to the grooves 11, the buffer solution 6 moves along the grooves 11 from the first storage portion 31 and is supplied to the cells 7 staying in the respective grooves 11. Some of the buffer solution 6 may move along the grooves 11 and reach the second storage portion 32 (second recess 14).

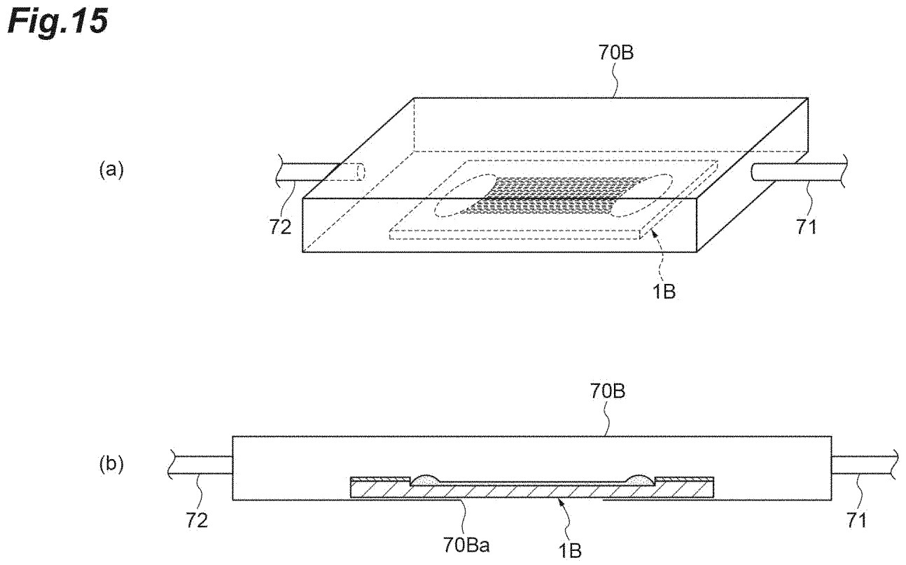

Subsequently, the exposure unit 1B is disposed in a gas sample chamber 70B as illustrated in FIGS. 15(a) and 15(b). An opening 70Ba is formed in the bottom surface of the gas sample chamber 70B. The exposure unit 1 is disposed in the gas sample chamber 70B so as to block the opening 70Ba. The opening 70Ba may not be formed in the gas sample chamber 70B. In this case, the part corresponding to the opening 70Ba may be configured to be transparent. Subsequently, observation is started in the same manner as in the first embodiment.

According to the second modification example as well as the first embodiment, the liquid layer of the buffer solution 6 covering the cells 7 can be kept at a suitable thickness when the gas-phase substances 5 are exposed to the cells 7 in the buffer solution 6. In addition, since the base body 2 is configured only by the glass substrate 10, the configuration can be simplified. In addition, the configuration can be simplified also by configuring the first storage portion 31 with the first recess 13 formed in the base body 2.

### Second Embodiment

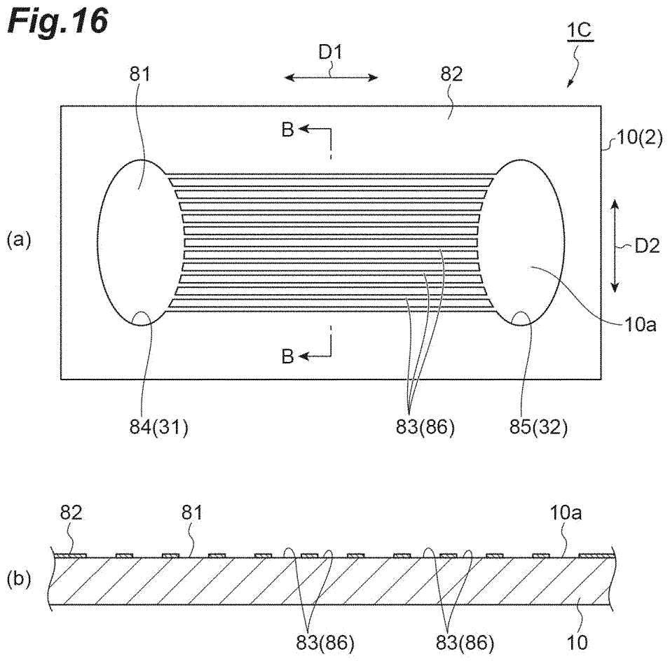

In an exposure unit 1C of the second embodiment illustrated in FIG. 16, the base body 2 has only the glass substrate 10 and does not have the rubber sheet 20. The surface 10a of the glass substrate 10 does not have the grooves 11. The surface 10a of the glass substrate 10 has a hydrophilic region 81 and a hydrophobic region 82 less hydrophilic than the hydrophilic region 81. In this example, the hydrophilic region 81 and the hydrophobic region 82 are formed by hydrophobically treating a part of the surface 10a. In other words, the hydrophobic region 82 is a hydrophobically treated region and the hydrophilic region 81 is a hydrophobic treatment-less region. For example, in the hydrophobic region 82, a layered hydrophobic pattern made of a hydrophobic coating is formed as the hydrophobic treatment.

The shape of the hydrophilic region 81 in a plan view is, for example, the same as the plan-view shape of the groove 11, the first recess 13, and the second recess 14 of the second modification example described above. The hydrophilic region 81 has a plurality of linear parts 83, a first elliptical part 84, and a second elliptical part 85. Each linear part 83 linearly extends along the first direction D1. The plurality of linear parts 83 are arranged at regular intervals in the second direction D2 orthogonal to the first direction D1. The plurality of linear parts 83 have the same shape.

In a plan view, each of the first elliptical part 84 and the second elliptical part 85 has, for example, an elliptical shape with a long axis along the second direction D2. Each of the first elliptical part 84 and the second elliptical part 85 is connected to each linear part 83.

By the surface 10a of the glass substrate 10 being partitioned into the hydrophilic region 81 and the hydrophobic region 82, the hydrophilic region 81 has a cell holding portion 86 where the cells 7 are retained together with the buffer solution 6, the first storage portion 31 for storing the buffer solution 6 to be supplied to the cell holding portion 86, and the second storage portion 32 for storing the buffer solution 6 discharged from the cell holding portion 86. In the second embodiment, the cell holding portion 86 is configured by the plurality of linear parts 83. The first elliptical part 84 configures the first storage portion 31, and the second elliptical part 85 configures the second storage portion 32.

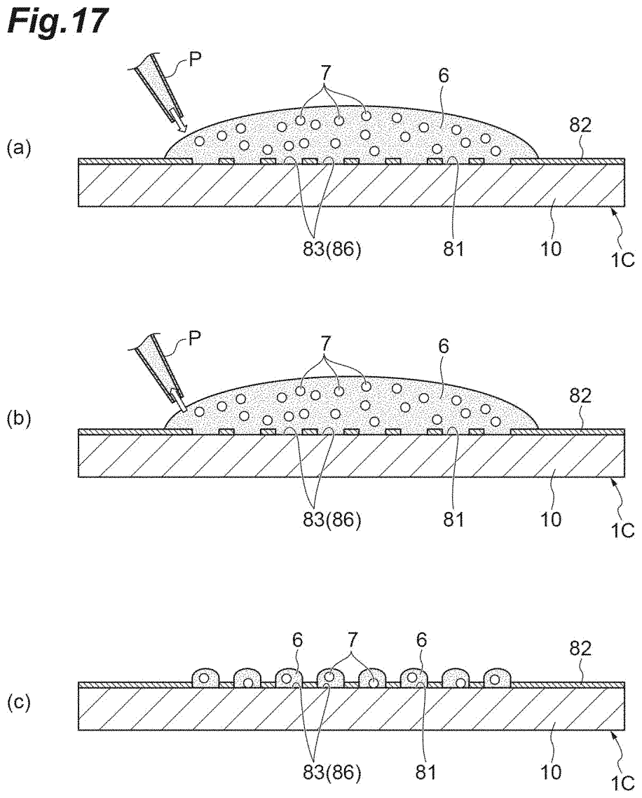

In an observation method of the second embodiment, as illustrated in FIG. 17(a), a suspension of the buffer solution 6 and the cells 7 is dripped around the cell holding portion 86 with the pipette P. Subsequently, as illustrated in FIG. 17(b), the dripped suspension is suctioned out with the pipette P and removed. At this time, surface tension causes some of the suspension to remain on the cell holding portion 86 as illustrated in FIG. 17(c). As a result, the cells 7 are seeded in the cell holding portion 86 (each linear part 83). The cells 7 on the cell holding portion 86 are covered with the buffer solution 6. In other words, the cells 7 are retained in the cell holding portion 86 together with the buffer solution 6, and a liquid layer of the buffer solution 6 covering the cells 7 is formed.

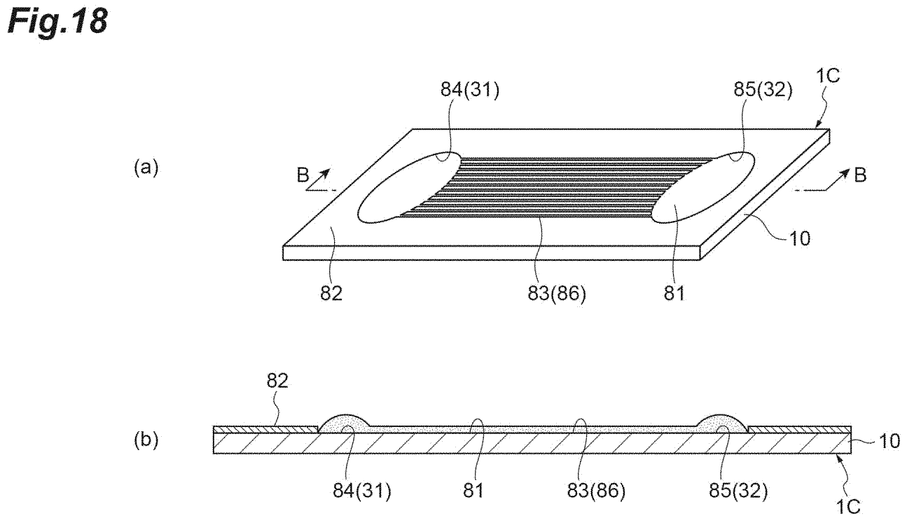

Subsequently, the buffer solution 6 is dripped into the first storage portion 31 (first elliptical part 84) as illustrated in FIGS. 18(a) and 18(b). Since the first storage portion 31 is connected to the cell holding portion 86, the buffer solution 6 moves along the grooves 11 from the first storage portion 31 and is supplied to the cells 7 staying in the cell holding portion 86. Some of the buffer solution 6 may move along the cell holding portion 86 and reach the second storage portion 32 (second elliptical part 85).

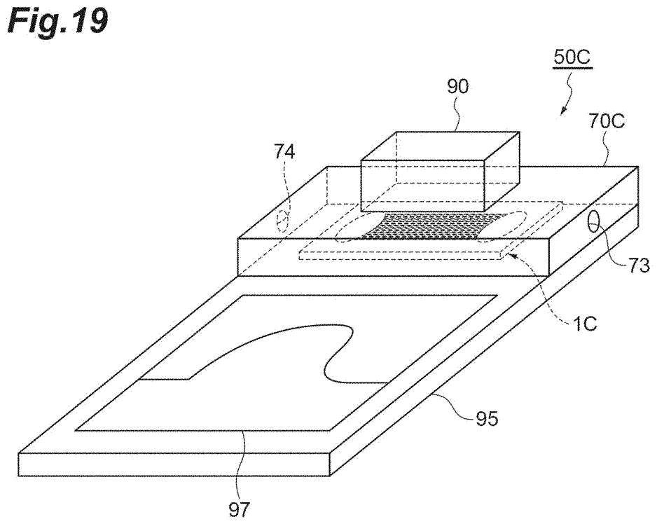

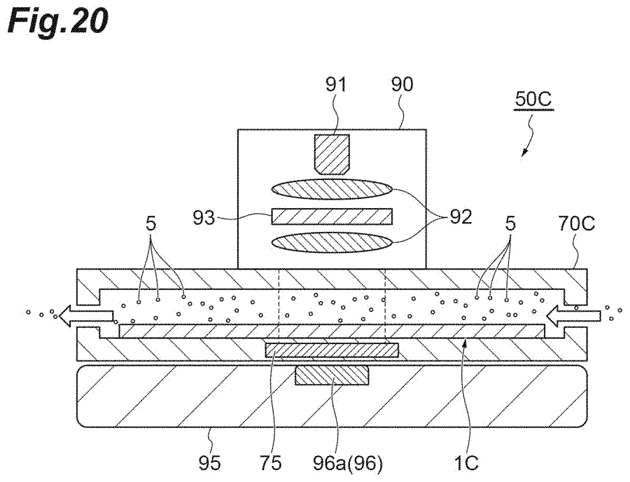

Subsequently, the exposure unit 1C is disposed in a gas sample chamber 70C as illustrated in FIGS. 19 and 20. An inflow port 73 for gas inflow is formed in one side surface of the gas sample chamber 70C, and a discharge port 74 for gas discharge is formed in the other side surface of the gas sample chamber 70C. An optical filter 75 transmitting the light L is disposed on the bottom surface of the gas sample chamber 70C. The optical filter 75 is, for example, a fluorescence-transmitting fluorescence filter.

An observation device 50C of the second embodiment includes an excitation optical system 90 disposed on the upper surface side of the gas sample chamber 70C. The excitation optical system 90 has a light source 91, a pair of lenses 92, and an excitation filter 93. The light source 91 outputs excitation light for exciting the cell 7. The excitation light output from the light source 91 passes through one of the lenses 92, the excitation filter 93, and the other lens 92 in this order, is transmitted through the upper surface of the gas sample chamber 70C, and enters the exposure unit 1C. The gas sample chamber 70C and the excitation optical system 90 are light-shielded such that external light does not enter.

The observation device 50C of the second embodiment includes a detection terminal 95 disposed on the bottom surface side of the gas sample chamber 70C. The detection terminal 95 is, for example, a portable terminal such as a smartphone. The detection terminal 95 has a light detection unit 96 and a display unit 97. The light detection unit 96 has a camera lens 96a and detects the light L from the cell 7. The display unit 97 displays, for example, a measurement result. The detection terminal 95 is disposed such that the camera lens 96a faces the optical filter 75. Subsequently, observation is started in the same manner as in the first embodiment. During the observation, a gas containing the substance 5 flows into the gas sample chamber 70C. The gas may flow in by diffusion or by a fan attached to the discharge port 74. In this manner, in the observation method (exposure method) using the observation device 50C, the gas-phase substance 5 is exposed to the cell 7 retained in the cell holding portion 86 in a state where the cell 7 is retained in the cell holding portion 86 together with the buffer solution 6 and the buffer solution 6 to be supplied to the cell holding portion 86 is stored in the first storage portion 31.

According to the exposure unit 1C of the second embodiment as well, when the gas-phase substances 5 are exposed to the cells 7 in the buffer solution 6, the liquid layer of the buffer solution 6 covering the cells 7 can be kept at a suitable thickness. That is, in the exposure unit 1C, when the suspension of the cells 7 and the buffer solution 6 is disposed in the cell holding portion 86 (linear part 83), the cells 7 are retained in the cell holding portion 86 together with the buffer solution 6 and a liquid layer of the buffer solution 6 covering the cells 7 is formed. The thickness of this liquid layer is a constant thickness in accordance with the configuration (for example, dimensions, hydrophilicity) of the cell holding portion 86. Accordingly, in the exposure unit 1C, the liquid layer can be kept thin. In addition, the hydrophilic region 81 has the first storage portion 31 (first elliptical part 84) connected to the cell holding portion 86 and storing the buffer solution 6 to be supplied to the cell holding portion 86. As a result, the buffer solution 6 can be supplied from the first storage portion 31 to the cell holding portion 86, and drying of the cells 7 can be prevented. As a result, according to the exposure unit 1C, when the gas-phase substance 5 is exposed to the cell 7 in the buffer solution 6, the liquid layer of the buffer solution 6 covering the cell 7 can be kept at a suitable thickness.

The present disclosure is not limited to the embodiments and modification examples described above. For example, the material and shape of each configuration are not limited to those described above, and various materials and shapes can be adopted. The first member is not limited to the glass substrate 10 and may be formed of another transparent or light-shielding material. The second member is not limited to the rubber sheet 20 and may be formed of another material.

Although the entire glass substrate 10 (first member) is configured to be transparent with respect to the light L in the first embodiment, it is sufficient that the part of the first member where the groove 11 is formed is configured to be transparent with respect to the light L, and the part where the groove 11 is not formed may have a light shielding property against the light L. The entire first member may be formed of a light-shielding material.

In the first embodiment, the hydrophobic pattern 12 may be omitted. In each example described above, the second storage portion 32 may be omitted. Although the first storage portion 31 is configured only by the second opening 22 in the first embodiment, the first storage portion 31 may be configured by the second opening 22 and a recess formed in the surface 10a of the glass substrate 10. Likewise, the second storage portion 32 may be configured by the third opening 23 and a recess formed in the surface 10a. The exposure units 1, 1A, 1B, and 1C described above can be used in a case where, for example, an atmospheric harmful substance is exposed to a cell as well as a case where an odor substance is exposed to an odor detection cell.

### REFERENCE SIGNS LIST

1, 1A, 1B, 1C: exposure unit, 2: base body, 5: substance, 6: buffer solution, 7: cell, 10: glass substrate (first member), 10a: surface, 11: groove, 11a: inner surface, lib: one end, 11c: the other end, 13: first recess, 20: rubber sheet (second member), 21: first opening, 22: second opening, 31: first storage portion, 32: second storage portion, 50, 50C: observation device, 60: optical system, 81: hydrophilic region, 82: hydrophobic region, 86: cell holding portion, L: light from cell.

1. An exposure unit used in order to expose a gas-phase substance to a cell in a buffer solution, the exposure unit comprising a base body,
wherein the base body includes:
a groove formed in the base body so as to be exposed to an outside and retaining the cell together with the buffer solution; and
a first storage portion connected to one end of the groove and storing the buffer solution to be supplied to the groove.

2. The exposure unit according to claim 1, wherein
the base body includes a first member having a surface where the groove is formed and a second member disposed on the surface,
a first opening and a second opening are formed in the second member, and
the groove is exposed to the outside via the first opening and at least a part of the first storage portion is configured by the second opening.

3. The exposure unit according to claim 2, wherein a plurality of the first openings are formed in the second member.

4. The exposure unit according to claim 1, wherein a part of the base body where the groove is formed is configured to be transparent with respect to light from the cell retained in the groove.

5. The exposure unit according to claim 1, wherein the base body further includes a second storage portion connected to the other end of the groove and storing the buffer solution discharged from the groove.

6. The exposure unit according to claim 1, wherein hydrophilicity of an inner surface of the groove is higher than hydrophilicity of a part of the base body adjacent to the groove.

7. The exposure unit according to claim 1, wherein the groove includes a meanderingly extending part.

8. The exposure unit according to claim 1, wherein the first storage portion is configured by a recess formed in the base body.

9. An observation device comprising:
the exposure unit according to claim 1;
an optical system through which light from the cell retained in the groove passes; and
a photodetector detecting the light that has passed through the optical system.

10. An exposure unit used in order to expose a gas-phase substance to a cell in a buffer solution, the exposure unit comprising a base body and a surface of the base body including a hydrophilic region and a hydrophobic region less hydrophilic than the hydrophilic region,
wherein, by partitioning the surface of the base body into the hydrophilic region and the hydrophobic region, the hydrophilic region includes a cell holding portion where the cell is retained together with the buffer solution and a storage portion connected to the cell holding portion and storing the buffer solution to be supplied to the cell holding portion.

11. An exposure method for exposing the gas-phase substance to the cell retained in the groove using the exposure unit according to claim 1 in a state where the cell is retained in the groove together with the buffer solution and the buffer solution to be supplied to the groove is stored in the first storage portion.

12. An exposure method for exposing the gas-phase substance to the cell retained in the cell holding portion using the exposure unit according to claim 10 in a state where the cell is retained in the cell holding portion together with the buffer solution and the buffer solution to be supplied to the cell holding portion is stored in the storage portion.
